(19) United States
(12) Patent Application Publication
(10) Pub. No.: US 2007/0292942 A1
(43) Pub. Date: Dec. 20, 2007

(54) Light Scattering Determination of Treatment Potencies

(76) Inventor: Satish Deshpande, Guelph (CA)

Correspondence Address: DAVID J. BREMER, 4921 N. DAMEN AVENUE, CHICAGO, IL, 60625, US

(21) Appl. No.: 11/767,665

(22) Filed: Jun. 25, 2007

Related U.S. Application Data
(63) Continuation-in-part of application No. 11/611,880, filed on Dec. 17, 2006.
Application No. 11/611,880 is a continuation of application No. 10/416,099, filed on May 6, 2003, now abandoned.
(60) Provisional application No. 60/265,761, filed on Feb. 1, 2001.

(30) Foreign Application Priority Data
Jan. 25, 2002 (US) PCT/US02/02132

(51) Int. Cl. C12M 1/34

(52) U.S. Cl. 435/288.7

(57) ABSTRACT

Potencies of treatments changing cells are determined by difference between angular intensity distributions of light scattered by treated cells and by untreated cells at least in a treatment region of interest angular range.

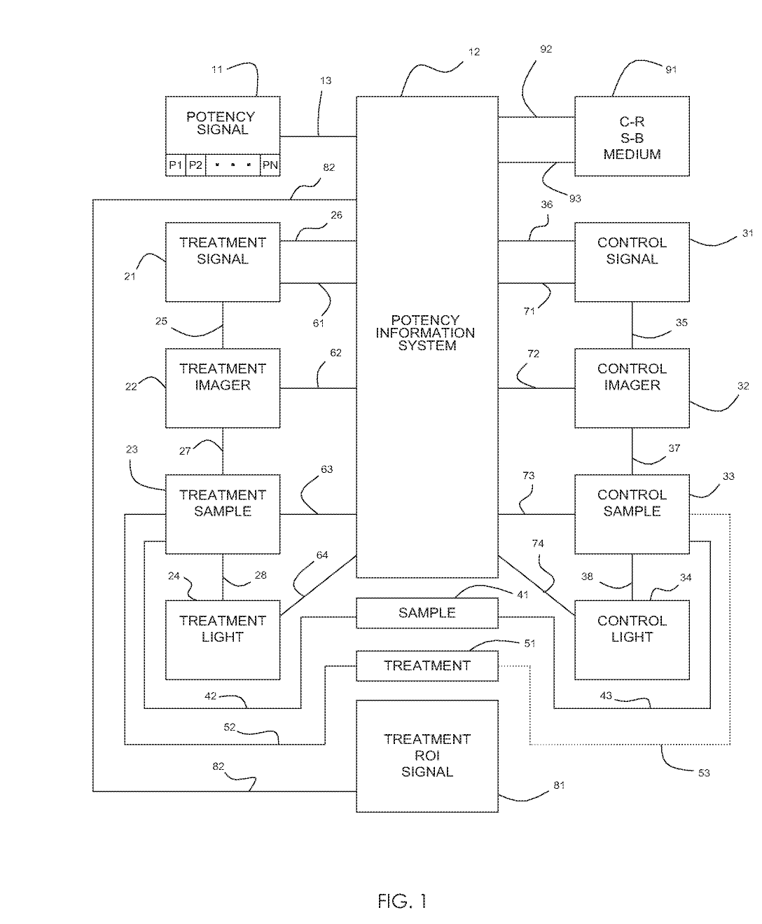

This application is a continuation in part of U.S. application Ser. No. 10/416,099 (confirmation number 1344) filed 6 May 2003 (published as US20040043433-A1 on 4 Mar. 2004), which is a 371 of PCT application PCT/US02/02132 filed 25 Jan. 2002 (published as WO02/077748 on 3 Oct. 2002), which claims benefit of U.S. provisional application 60/265,761 filed 1 Feb. 2001, all of which are incorporated in full here by reference.

The light scattering product for determining potencies of treatments on cell parts comprises a potency information system 12 which outputs 13 a potency signal 11, where the potency signal represents efficacy of a treatment 51 for changing a cell part in cells from a sample of cells 41. The light scattering can be in a treatment angular range where light is scattered by the cell part changed by the treatment. The samples can be scatterer optimized to get optimum peaks in the light scattered by the cell part changed by the treatment.

The invention provides progress over prior art because it solves a problem by ways and means which the prior art—individually and in combination—does not apply to the problem. The invention solves the problem by an unexpected discovery.

The invention is based on the discovery—not expected by others—that the potency of a treatment for a person with HIV infection can be determined by comparing scattered light angular intensity distributions from a sample of blood from the person and from a sample of that blood with a treatment added.

That the prior art—individually and in combination—teaches away from ways and means to make this discovery is clear evidence that the discovery was not expected by others skilled in the relevant art.

That alternate ways and means to solve the problem are much less economical is clear evidence that the discovery was not expected by others skilled in the relevant art.

Though the invention was made for application to the HIV/AIDS problem, it was found to be applicable in other cases where some agent is expected to change a cell part. Thus it solves serious problems in many areas, which is more clear evidence that the invention was not anticipated, was outside the scope of prior art, and was not obvious.

### DRAWINGS

The sole FIGURE graphically depicts light scattering determination of treatment potencies.

The potency signal is caused via the potency information system by at least a treatment signal 21 and a control signal 31.

The potency signal can have components (P1, P2, . . . Pn in 11) which represent efficacy of the treatment for changing the cell part in various forms.

The potency signal can be caused via the potency information system by at least a treatment signal 26, a control signal 36, and a treatment region of interest signal 81.

The control signal represents a control angular intensity distribution of control scattered light 37 which is a data set comprising intensities of light scattered by the control sample at various values of the opening angle between the central ray of the incident control light and light scattered by the control sample. The control scattered light is control light 34 incident on 38 and scattered by the cell part in a control sample 33. The control sample is from 43 the sample of cells.

The treatment region of interest signal 81 represents a treatment region of interest angular range over which the control angular intensity distribution comprises control light scattered by a cell part which is expected to be changed by the treatment. The treatment region of interest signal is signal-connected 82 with the potency information system.

The control sample can be control scatterer optimized. Control scatterer optimized is such that the number of control scatterers in the control sample which scatter control light into the treatment region of interest angular range is within a control scatterer optimum range. The control scatterer optimum range is such that lower numbers of control scatterers than the control scatterer optimum range will produce lower peaks in the control intensity distribution of control scattered light in the treatment region of interest angular range than produced by numbers of control scatterers within the control scatterer optimum range. The control scatterer optimum range is such that higher numbers of control scatterers than the control scatterer optimum range will produce broader peaks in the control intensity distribution of control scattered light in the treatment region of interest angular range than produced by control scatterers within the control scatterer optimum range.

The control scattered light is detected by a control imager 32. The control signal 31 is output 35 by the control imager and is signal-connected 36 with the potency information system.

The treatment signal represents a treatment angular intensity distribution of treatment scattered light which is a data set comprising intensities of light scattered by the treatment sample at various values of the opening angle between the central ray of the incident treatment light and light scattered by the treatment sample. The treatment scattered light is treatment light 24 incident on 28 and scattered by the cell part in a treatment sample 23. The treatment sample is from 42 the sample of cells 41 with the treatment 51 added 52.

The treatment sample can be treatment scatterer optimized. Treatment scatterer optimized is such that the number of treatment scatterers in the treatment sample which scatter treatment light into the treatment region of interest angular range is within a treatment scatterer optimum range. The treatment scatterer optimum range is such that lower numbers of treatment scatterers than the treatment scatterer optimum range will produce lower peaks in the treatment intensity distribution of treatment scattered light in the treatment region of interest angular range than produced by numbers of treatment scatterers within the treatment scatterer optimum range. The treatment scatterer optimum range is such that higher numbers of treatment scatterers than the treatment scatterer optimum range will produce broader peaks in the treatment intensity distribution of treatment scattered light in the treatment region of interest angular range than produced by treatment scatterers within the treatment scatterer optimum range.

The treatment scattered light is detected 27 by a treatment imager. The treatment signal 21 is output 25 by the treatment imager and signal-connected 26 with the potency information system.

The product can also comprise several components of the potency information system:

A control light component of the potency information system is signal-connected 74 with the control light and can vary properties of the control light.

A treatment light component of the potency information system is signal-connected 64 with the treatment light and can vary properties of the treatment light.

A control imager component of the potency information system is signal-connected 72 with the control imager and can vary properties of the control imager.

A treatment imager component of the potency information system is signal-connected 62 with the treatment imager and can vary properties of the treatment imager.

A control signal component of the potency information system is signal-connected 71 with the control signal and can vary output of the control signal.

A treatment signal component of the potency information system is signal-connected 61 with the treatment signal and can vary output of the treatment signal.

A control sample component of the potency information system is signal-connected 73 with the control sample and—when the control sample is a first control sample member of a control sample plurality of control samples, where the first control sample member is from the sample—can cause exchange to make a second control sample member of the control sample plurality the control sample, where the second control sample member is from the sample of cells.

A treatment sample component of the potency information system is signal-connected 63 with the treatment sample and—when the treatment is a first member of a treatment plurality of treatments and the treatment sample is a first treatment sample member of a treatment sample plurality of treatment samples, where the first member of the treatment sample plurality is from the sample of cells with the first member of the treatment plurality added—can cause exchange to make the treatment sample a second member of the treatment sample plurality where the second member of the treatment sample plurality is from the sample of cells with a second member of the treatment plurality.

The control sample component and the treatment sample component allow for a plurality of control sample and treatment sample pairs to be imaged serially and in parallel. These components also allow for investigation of accumulation effects of treatments; such as, first comparing a first control sample with a first treatment sample, then using 53 the first treatment sample as a second control sample which is compared with the a second treatment sample which is the first treatment sample with a second treatment added.

A computer-readable, signal-bearing medium 91 can be signal-connected 92, 93 with the potency information system.

To get the best potency determination a treatment region of interest angular range is determined. Control light scattered into the treatment region of interest angular range comprises light scattered by the cell part which the treatment is expected to change. The treatment region of interest angular range is represented by the treatment region of interest signal 81 which is signal-connected 82 with the potency information system.

For example, there is a peak at about 0.66 degrees in the control angular intensity distribution comprising light scattered by a person's white blood cell nuclei. The potency of an HIV/AIDS treatment expected to change these cell nuclei is best determined by changes in a small angular range bracketing this peak.

In this example, an HIV/AIDS treatment expected to change these cell nuclei, which causes a large change in the zero to four degree range, but causes a small change in the peak bracketing 0.66 degree peak, is not as potent as cell-nuclei-changing treatments which cause greater changes in the treatment region of interest angular range bracketing the 0.66 degree peak.

The treatment region of interest angular range can vary from person to person, depending for example in the HIV/AIDS case on viral load.

It is equally important to identify a treatment angular range and use a treatment region of interest signal in other cases such as determining the potency of treatments against other cell conditions such as cancers.

In cases where it is not known which cell part a treatment changes, the product can indicate which cell part is changed by the angular range where there is greater difference between the control signal and the treatment signal.

Also, to get the best potency determination the control sample is control scatterer optimized and the treatment sample is treatment scatterer optimized. The control scatterer optimum range depends on the concentration of scatterers, on the cross section of the control light, and on the thickness of the control sample, and can be determined by experiment. The treatment scatterer optimum range depends on the concentration of scatterers, on the cross section of treatment light, and on the thickness of the treatment sample, and can be determined by experiment. In most cases the treatment scatterer optimum range and the control scatterer optimum range will be the same.

The potency signal can have various potency signal components. A component can be a correlation coefficient between a control angular intensity distribution and a treatment angular intensity distribution at least over the treatment angular range. A component can be a correlation coefficient between a control angular intensity distribution and a treatment angular intensity distribution over an angular range larger than the treatment angular range.

A component can be a time series of correlation coefficients between a control angular intensity distribution and a treatment angular intensity distribution at least over the treatment angular range. A component can be a time series of correlation coefficients between a control angular intensity distribution and a treatment angular intensity distribution over an angular range larger than the treatment angular range. The time-series potency components can represent the kinetics of changes caused by the treatment.

The equation 1/q=λ/(4πη)1/sin(θ/2) can be used to transform angular intensity distributions to length intensity distributions, where 1/q is a length related to the size of the cell part scattering the light of wavelength λ at angle θ, and η is the index of refraction of the cell part. (The index of refraction of the control sample as a whole can be used here.) Correlation coefficient—and time series thereof—components of the potency signal can be formed using these length intensity distributions.

A potency signal component can comprise a moving correlation coefficient calculation where, for example, when subsequent imager pixels are at subsequent angles away from the central ray, a first correlation coefficient between the control signal and the treatment signal is calculated for pixels one to N, a second correlation coefficient between the control signal and the treatment signal for pixels two to N+1, and so forth through the pixels recorded. The lowest value in this set of correlation coefficients in the treatment angular range indicates the potency of the treatment.

Potency signal components can comprise display and printed plots of the control and treatment intensity distributions. Potency signal components can be point-by-point subtractions of a treatment intensity distribution from a control intensity distribution in the treatment angular range and in larger angular ranges. Potency signal components can be comparisons of areas under intensity distribution curves in the treatment angular range and larger angular ranges.

The potency signal can comprise any one of potency signal components, can comprise a subset of any of the potency signal components, and can comprise all of the potency signal components.

Any means and ways—and combinations of means and ways—for representing changes between a control intensity distribution and a treatment intensity distribution can be used to form a component of the potency signal. The best means and ways for a specific type of cell, specific type of cell condition, and specific type of treatment can be determined by experiment.

Experiments show that treatments which cause the greatest potency signals have the greatest treatment potency. For example, experiments show that—for a sample prepared from any specific HIV infected blood donor—the HIV treatment which causes the greatest potency signal has the greatest potency against HIV for that blood donor, where the HIV infection can be by various pluralities of the various forms of HIV.

The control light and the treatment light can be from two separate light sources, can be from the one light source, and can be from portions of one light source. The control imager and the treatment imager can be two separate imagers, can be one imager, and can be portions of one imager. An imager—and imagers, and portions of an imager—can detect the control scattered light and the treatment scattered light concurrently using beam splitting means.

The control scattered light and the treatment scattered light must be comparable. This can be achieved in various ways. For example, the conditions of the two imagings can be interchangeable, except for the added treatment, so that the control scattered light and the treatment scattered light are directly comparable. For example, the two imagings can be calibrated with a standard imaging, so that the control scattered light and the treatment scattered light are comparable via the calibrations.

Various imagers using various imaging means can be used. Scattered light can illuminate a screen which can be imaged. Scattered light can be imaged directly.

Useful results are obtained using commercial video cameras for the imagings. Here imaging means need not form an image. The imager can be any means for detecting an angular intensity distribution of scattered light.

The control sample comprises cells from a sample of cells. For example, the sample can be white blood cells from a person infected with a human immunodeficiency virus and can be white blood cells from a person infected with several variations of the human immunodeficiency virus.

The treatment sample comprises cells from the sample with a treatment added. The treatment can be one of a plurality of treatments expected to change cells from the sample.

A “treatment” here—and throughout—means any substance which causes changes in parts of cells from the sample. The changes can be to surface parts of cells, to interior parts of cells, and to combinations of these. The changes can be known changes and can be unknown changes.

The computer-readable signal-bearing medium 93 can have an historical component which can comprise representations of historical control signals and can comprise representations of historical treatment signals. “Representations” can be various kinds of averages of historical data, constructions from historical data according to some criteria, bellwether data culled from historical data, and combinations of these.

Historical data can be used in the calculation of the potency signal. Historical data can be used in causing the control signal and the treatment signal. Historical data can be used in configuring the imagers and in preparing the samples. In some cases historical data can be the control signal.

The product comprises the elements and connections specified here and comprises the method by which these elements and connections are used to determine potencies of treatments on cells.

Thus, the product also comprises preparing a sample of cells, preparing a control sample of cells, preparing a treatment sample of cells, illuminating the control sample with control light, illuminating the treatment sample with treatment light, detecting control scattered light with a control imager, detecting treatment scattered light with a treatment imager, and outputting a potency signal which represents difference between a treatment angular intensity distribution and a control angular intensity distribution by means and ways which produce the potency signal.

The product does not depend on how control and treatment samples are prepared, on how the angular scattering intensity distributions are obtained, nor on how difference between the distributions are determined.

The product can use various means for preparing control and treatment samples and can use various elements and configurations to obtain the angular scattering intensity. The product can be used with any means and ways for detecting angular intensity distributions and comparing the results.

A “signal” from a product part to a second product part—and a product part being “signal-connected” with a second product part—here, and throughout, mean that a physical state of the product part causes a second physical state of the second product part. This can occur by various direct causal means and can occur by any of various transmission means. Transmitted signals can be any of various point-to-point and broadcast forms of energy transmission such as wireless and via wires, cables, and fibers. Parts of transmitted signals can reside with one form of the transmitted signal, parts can reside with a second form of transmitted signal, and parts can reside with various combinations of transmitted signals.

The several causes here can act via any of various processing modes. The processing can utilize configured processing elements such as fixed circuits, can utilize configurable processing elements such as field programmable gate arrays and neural networks, can utilize instructions in a data-bearing medium, and can utilize combinations of these. The processing can be by stand alone means, can act via a local information system, can act via a networked information system, and can act via combinations of these. The processing—in part at least—can be by parts of an imager. The computer-readable signal-bearing medium can be a transmitted signal, a data storage medium, and a combination of a transmitted signal and a data storage medium.

Claimed is:

1. A light scattering product for determining potencies of treatments on cell parts, the product comprising:
a potency information system which outputs a potency signal,
the potency signal representing efficacy of a treatment for changing a cell part in cells from a sample of cells,
the potency signal being caused via the potency information system by at least a treatment signal and a control signal,
the control signal representing a control angular intensity distribution of control scattered light,
the control scattered light being control light scattered by the cell part in a control sample,
the control sample being from the sample of cells,
the control scattered light being detected by a control imager,
the control signal being output by the control imager and being signal-connected with the potency information system,
the treatment signal representing a treatment angular intensity distribution of treatment scattered light,
the treatment scattered light being treatment light scattered by the cell part in a treatment sample,
the treatment sample being from the sample of cells with the treatment added,
the treatment scattered light being detected by a treatment imager,
the treatment signal being output by the treatment imager and signal-connected with the potency information system.

2. A light scattering product for determining potencies of treatments on cell parts, the product comprising:
a potency information system which outputs a potency signal,
the potency signal representing efficacy of a treatment for changing a cell part in cells from a sample of cells,
the potency signal being caused via the potency information system by at least a treatment signal, a control signal, and a treatment region of interest signal,
the control signal representing a control angular intensity distribution of control scattered light,
the control scattered light being control light scattered by the cell part in a control sample,
the control sample being from the sample of cells,
the control scattered light being detected by a control imager,
the control signal being output by the control imager and being signal-connected with the potency information system,
the treatment signal representing a treatment angular intensity distribution of treatment scattered light,
the treatment scattered light being treatment light scattered by the cell part in a treatment sample,
the treatment sample being from the sample of cells with the treatment added,
the treatment scattered light being detected by a treatment imager,
the treatment signal being output by the treatment imager and signal-connected with the potency information system;
the treatment region of interest signal representing a treatment region of interest angular range over which the control angular intensity distribution comprises control light scattered by a cell part which is expected to be changed by the treatment,
the treatment region of interest signal being signal-connected with the potency information system; and
a computer-readable, signal-bearing medium signal-connected with the potency information system.

3. A light scattering product for determining potencies of treatments on cell parts, the product comprising:
a potency information system which outputs a potency signal,
the potency signal representing efficacy of a treatment for changing a cell part in cells from a sample of cells,
the potency signal being caused via the potency information system by at least a treatment signal, a control signal, and a treatment region of interest signal,
the control signal representing a control angular intensity distribution of control scattered light,
the control scattered light being control light scattered by the cell part in a control sample,
the control sample being from the sample of cells,
the control scattered light being detected by a control imager,
the control signal being output by the control imager and being signal-connected with the potency information system,
the treatment signal representing a treatment angular intensity distribution of treatment scattered light,
the treatment scattered light being treatment light scattered by the cell part in a treatment sample,
the treatment sample being from the sample of cells with the treatment added,
the treatment scattered light being detected by a treatment imager,
the treatment signal being output by the treatment imager and signal-connected with the potency information system;
the treatment region of interest signal representing a treatment region of interest angular range over which the control angular intensity distribution comprises control light scattered by a cell part which is expected to be changed by the treatment,
the treatment region of interest signal being signal-connected with the potency information system;
the control sample being control scatterer optimized,
control scatterer optimized being such that that the number of control scatterers in the control sample which scatter control light into the treatment region of interest angular range is within a control scatterer optimum range,
the control scatterer optimum range being such that lower numbers of control scatterers than the control scatterer optimum range will produce lower peaks in the control intensity distribution of control scattered light in the treatment region of interest angular range than produced by numbers of control scatterers within the control scatterer optimum range,
and the control scatterer optimum range being such that higher numbers of control scatterers than the control scatterer optimum range will produce broader peaks in the control intensity distribution of control scattered light in the treatment region of interest angular range than produced by control scatterers within the control scatterer optimum range.
the treatment sample being treatment scatterer optimized,
treatment scatterer optimized being such that that the number of treatment scatterers in the treatment sample which scatter treatment light into the treatment region of interest angular range is within a treatment scatterer optimum range,
the treatment scatterer optimum range being such that lower numbers of treatment scatterers than the treatment scatterer optimum range will produce lower peaks in the treatment intensity distribution of treatment scattered light in the treatment region of interest angular range than produced by numbers of treatment scatterers within the treatment scatterer optimum range,
and the treatment scatterer optimum range being such that higher numbers of treatment scatterers than the treatment scatterer optimum range will produce broader peaks in the treatment intensity distribution of treatment scattered light in the treatment region of interest angular range than produced by treatment scatterers within the treatment scatterer optimum range; and
a computer-readable, signal-bearing medium signal-connected with the potency information system.

4. The product of claim 3 wherein the potency signal has components.

5. The product of claim 3 further comprising:
a control light component of the potency information system, which is signal-connected with the control light and can vary properties of the treatment light;
a treatment light component of the potency information system, which is signal-connected with the treatment light and can vary properties of the treatment light;
a control imager component of the potency information system, which is signal-connected with the control imager and can vary properties of the control imager;
a treatment imager component of the potency information system, which is signal-connected with the treatment imager and can vary properties of the treatment imager;
a control signal component of the potency information system, which is signal-connected with the control imager and can vary output of the control signal;
a treatment signal component of the potency information system, which is signal-connected with the treatment imager and can vary output of the treatment signal;
a control sample component of the potency information system, which is signal-connected with the control sample, and which—when the control sample is a first control sample member of a control sample plurality of control samples, where the first control sample member is from the sample—can cause exchange to make a second control sample member of the control sample plurality the control sample, where the second control sample member is from the sample of cells; and
a treatment sample component of the potency information system, which is signal-connected with the treatment sample, and which—when the treatment is a first member of a treatment plurality of treatments and the treatment sample is a first treatment sample member of a treatment sample plurality of treatment samples, where the first member of the treatment sample plurality is from the sample of cells with the first member of the treatment plurality added—can cause exchange to make the treatment sample a second member of the treatment sample plurality where the second member of the treatment sample plurality is from the sample of cells with a second member of the treatment plurality added.
